(19) United States
(12) Patent Application Publication
(10) Pub. No.: US 2024/0132851 A1
(43) Pub. Date: Apr. 25, 2024

(54) Methods for Differentiating Endothelial Cells

(71) Applicant: Lung Biotechnology PBC, Silver Spring, MD (US)

(72) Inventors: Adrian COOPER, Silver Spring, MD (US); Andre DHARMAWAN, Silver Spring, MD (US); Elena FEDERZONI, Silver Spring, MD (US); Kurt WONG, Silver Spring, MD (US)

(21) Appl. No.: 18/354,212

(22) Filed: Jul. 17, 2023

Related U.S. Application Data
(60) Provisional application No. 63/390,445, filed on Jul. 19, 2022.

(51) Int. Cl. C12N 5/071; C12N 5/00

(52) U.S. Cl. CPC C12N 5/069; C12N 5/0018; C12N 5/0068; C12N 5/0081; C12N 2500/02; C12N 2501/115; C12N 2501/155; C12N 2501/165; C12N 2501/999; C12N 2506/02; C12N 2506/45; C12N 2513/00; C12N 2533/54

(57) ABSTRACT

The present disclosure relates to methods of differentiating pluripotent stem cells into endothelial cells. The present disclosure also relates to endothelial cells made by such methods, organoids containing such endothelial cells, and methods of use thereof. The present disclosure also relates to differentiation media for use in the same.

### CROSS REFERENCE TO RELATED APPLICATIONS

This application claims the priority benefit of U.S. Provisional Application No. 63/390,445, filed Jul. 19, 2022, which is incorporated herein by reference in its entirety.

### FIELD OF THE DISCLOSURE

The present disclosure relates to methods of differentiating pluripotent stem cells into endothelial cells. The present disclosure also relates to endothelial cells made by such methods, organoids containing such endothelial cells, and methods of use thereof. The present disclosure also relates to differentiation medium for use in the same.

### BACKGROUND

Pluripotent stem cells (PSC) are undifferentiated or partially differentiated cells that can differentiate into various other cell types. Induced pluripotent stem cells (iPSC) are a type of PSC derived from adult somatic cells that have been genetically reprogrammed to an embryonic stem cell (ESC)-like state through the expression of genes and factors important for maintaining the defining properties of ESC. iPSC have generated interest in the medical community recently because they address many obstacles associated with the use of embryonic stem cells, and allow for the generation of patient-specific PSC, which can be genetically corrected, differentiated into adult lineages, and returned to the same patient as an autograft. Yamanaka et al., Cell Stem Cell. 1(1):39-49 (2007); Nishikawa et al., Nat. Rev. Mol. Cell Biol. 9:725 (2008). In addition to genetic disorders, iPSC can be used for tissue regeneration and disease modeling. Kogut et al., Methods Mol. Biol. 1195:1-12 (2014). PSC and iPSC can be differentiated into many different cell types, including endothelial cells (EC). Jang et al., Am. J. Pathol. 189(3):502-512 (2019); Gu et al., Curr. Protoc. Hum. Genet. published online 2018 Jul. 6. doi: 10.1002/cphg.64.

### BRIEF SUMMARY

The present disclosure provides methods for differentiating pluripotent stem cells (PSC) into endothelial cells (EC).

In some aspects, the method comprises (i) culturing PSC (e.g., iPSC) on a collagen IV-coated surface in a base culture medium comprising a Rho-associated, coiled-coil containing protein kinase (ROCK) inhibitor; (ii) culturing the cells of (i) on a collagen IV-coated surface in a base culture medium comprising a glycogen synthase kinase 3 (GSK3) inhibitor; (iii) culturing the cells of (ii) on a collagen IV-coated surface in a base culture medium comprising fibroblast growth factor 2 (FGF2), vascular endothelial growth factor (VEGF), and bone morphogenetic protein 4 (BMP4) for about 4 days; (iv) culturing the cells of (iii) on a collagen IV-coated surface in a base culture medium comprising FGF2 and VEGF for about 2 days, wherein the culture medium does not comprise BMP4; and (v) separating the cells of (iv) having expression of CD144 to form endothelial cells.

In some aspects, the method further comprises (vi) culturing the cells of (v) having expression of CD144 in a base culture medium comprising a transforming growth factor β (TGFβ) inhibitor.

In some aspects, the method comprises (i) culturing PSC (e.g., iPSC) on a collagen IV-coated surface in a base culture medium comprising a ROCK inhibitor; (ii) culturing the cells of (i) on a collagen IV-coated surface in a base culture medium comprising a GSK3 inhibitor; (iii) culturing the cells of (ii) on a collagen IV-coated surface in a base culture medium comprising FGF2, VEGF, and BMP4; (iv) separating the cells of (iv) having expression of CD144; and (v) culturing the cells of (v) having expression of CD144 on a collagen I-coated surface in a base culture medium comprising a TGFβ inhibitor to form endothelial cells.

In some aspects, the ROCK inhibitor is Y-27632. In some aspects, Y-27632 is present in the culture medium at a concentration of about 10 μM.

In some aspects, the culturing of (i) is for about 1 day.

In some aspects, the GSK3 inhibitor is CHIR99021. In some aspects, CHIR99021 is present in the culture medium at a concentration of about 36 μM.

In some aspects, the culturing of (ii) is for about 1 day.

In some aspects, FGF is present in the culture medium at a concentration of about 50 μg/mL.

In some aspects, VEGF is present in the culture medium at a concentration of about 50 μg/mL.

In some aspects, BMP4 is present in the culture medium at a concentration of about 50 μg/mL.

In some aspects, the cells are passaged between (iii) and (iv).

In some aspects, the culturing of (iii) is from about 4 days to about 6 days.

In some aspects, the TGFβ inhibitor is SB431542. In some aspects, SB431542 is present in the culture medium at a concentration of about 10 μM.

In some aspects, the culturing of (vi) is for about 6 days. In some aspects, the culturing of (v) is for about 6 days.

In some aspects, the separating of (iv) is by immunomagnetic cell separation. In some aspects, the separating of (v) is by immunomagnetic cell separation.

In some aspects, the culturing of (i) and/or (ii) are performed under hypoxia conditions.

The present disclosure also provides endothelial cells made by a differentiation method disclosed herein, an organoid containing endothelial cells disclosed herein, and certain methods of use thereof.

The present disclosure also provides differentiation media for use in the same.

### BRIEF DESCRIPTION OF THE DRAWINGS

Some aspects of the invention are herein described, by way of example only, with reference to the accompanying drawings. With specific reference now to the drawings in detail, it is stressed that the particulars shown are by way of example and for purposes of illustrative discussion of aspects of the invention.

FIG. 1 is a schematic of the differentiation protocol described in Example 1.

FIG. 2 shows BJRiPS-EC differentiation flow cytometry data described in Example 1.

FIG. 3A is an exemplary whole-cell image from day 0 of the differentiation protocol described in Example 1.

FIG. 3B is an exemplary whole-cell image from day 2 of the differentiation protocol described in Example 1.

FIG. 3C is an exemplary whole-cell image from day 5 of the differentiation protocol described in Example 1.

FIG. 3D is an exemplary whole-cell image from day 9 after CD144+ selection of the differentiation protocol described in Example 1.

FIG. 3E is an exemplary whole-cell image from day 13 after CD144+ selection of the differentiation protocol described in Example 1.

FIG. 3F is an exemplary whole-cell image from day 17 after CD144+ selection of the differentiation protocol described in Example 1.

FIG. 4A shows progression to endothelial cell (EC) phenotype by SSEA4 flow cytometry results from day 6, day 10, day 13, day 19 and day 26 of the differentiation protocol described in Example 1.

FIG. 4B shows progression to EC phenotype with CD140-flow cytometry results from day 6, day 10, day 13, day 19 and day 26 of the differentiation protocol described in Example 1.

FIG. 4C shows progression to EC phenotype with CD90-flow cytometry results from day 6, day 10, day 13, day 19 and day 26 of the differentiation protocol described in Example 1.

FIG. 5A-5C shows flow cytometry results for CD140b and CD90 surface markers in BJRiPs (iPSC; FIG. 5A), human pulmonary artery endothelial cells (HPAEC (primary EC; FIG. 5B) and test differentiated cells (iPS-EC, day 13; FIG. 5C) as described in Example 1.

FIG. 5D-5F shows flow cytometry results for CD31 and SSEA-4 surface markers in BJRiPs (iPSC; FIG. 5D), HPAEC (primary EC; FIG. 5E), and test differentiated cells (iPS-EC, day 13; FIG. 5F) as described in Example 1.

FIG. 6A-6B shows expression profiles of CD31 (FIG. 6A) and SSEA-4 (FIG. 6B) surface markers in BJRiPs (iPSC), HPAEC (primary EC) and test differentiated cells (iPS-EC; day 13) as described in Example 1.

FIG. 7A-7B shows tube formation results from test differentiated cells of Example 1 (FIG. 7A) and fibroblasts (FIG. 7B).

FIG. 8A-8B shows exemplary images of Ac-LDL assay results from test differentiated cells of Example 1 (FIG. 8A) and fibroblasts (FIG. 8B).

### DETAILED DESCRIPTION

### I. General Definitions

Unless defined otherwise, all technical and scientific terms used herein have the same meaning as commonly understood by one of ordinary skill in the art to which this disclosure belongs. In case of conflict, the present application, including the definitions, will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

### II. Differentiation Methods

The present disclosure relates to methods of differentiating pluripotent stem cells (PSC, e.g., iPSC) into endothelial cells (EC). Such methods result, for example, in improved endothelial cell viability, yield, and/or differentiated characteristics.

As used herein, the terms “differentiation” and “differentiating” refer to the process of inducing or reprogramming young or immature cells (e.g., pluripotent stem cells) into more mature or specialized cells (e.g., endothelial cells). In general, differentiation of pluripotent stem cells can be effected, for example, by changing culturing conditions of the cells, such as changing the stimuli agents in a culture medium or the physical state of the cells.

As used herein, the terms “pluripotent stem cell,” “pluripotent stem cells,” and “PSC” refer to young or immature cell(s) that can develop into more mature or specialized cells (e.g., endothelial cells).

In some aspects, PSC include, but are not limited to, embryonic stem cells (ESC), induced pluripotent stem cells (iPSC), embryonic germ cells, adult stem cells, or a combination thereof. In some aspects, the PSC are from a human. In some aspects, the PSC are from an animal. In some aspects, the animal is a sheep, pig or primate.

As used herein, the terms “induced pluripotent stem cell,” “induced pluripotent stem cells,” and “iPSC” refer to cells produced from differentiated adult, neonatal or fetal cells that have been induced or reprogrammed into pluripotent stem cells.

As used herein, the terms “endothelial cell,” “endothelial cells,” or “EC” refer to a cell or group of cells that form a single cell layer which lines blood vessels and regulate exchanges between the bloodstream and the surrounding tissues. As used herein, an endothelial cell includes a mature endothelial cell, an endothelial progenitor cell, and an endothelial precursor cell.

Endothelial cells generated by a differentiation method provided herein have one or more biochemical, functional, or morphological characteristics of an endothelial cell. A biochemical characteristic of an endothelial cell includes, but is not limited to, the ability to express one or more endothelial cell markers. Endothelial cell markers include, but are not limited to, vascular endothelial (VE)-cadherin (CD144), ACE (CD143), BNH9/BNF13, CD31, CD34, CD54 (ICAM-1), CD62E, CD105, CD146, Endocan (ESM-1), Endoglyx-1, Endomucin, Eotaxin-3, EPAS1, Factor VIII related antigen, FLI-1, Flk-1 (KDR, VEGFR-2), FLT-1 (VEGFR-1), GATA2, GBP-1, GRO-alpha, HEX, ICAM-2, LMO2, LYVE-1, MRB, Nucleolin, PAL-E, RTKs, sVCAM-1, TALI, TEM1, TEMS, TEM7, Thrombomodulin (TM, CD141), VCAM-1 (CD106), VEGF, vWF, ZO-1, ESAM, CD102, CD93, CD184, CD304, DLL4, and a tight junction protein (e.g., Claudin 5 or ZO-1).

Functional characteristics of an endothelial cell include, but are not limited to, the ability to take up acetylated low-density lipoprotein (ac-LDL); having barrier function; and the ability to respond to one or more pro-inflammatory stimuli (e.g., TNF, and IL-1) by upregulating the expression of cell adhesion molecules (e.g., CD54 (ICAM-1), CD106, and CD62E).

Morphological characteristics of an endothelial cell include, but are not limited to, the ability to form tube-like structures in a three-dimensional matrix, having a flattened (or squamous) appearance, and having a large, central nucleus.

Biochemical, functional and morphological characteristics of an endothelial cell can be readily determined by visual inspection or methods known in the art and described herein.

In some aspects, the differentiation methods provided herein include certain cell culturing conditions, such as culturing cells in certain culture medium.

As used herein, the terms “cell culture,” “cell culturing,” “culture,” “culturing,” and “cultured” refer to the maintenance, growth and/or differentiation of cells in an in vitro environment. The terms “cell culture medium,” “cell culture media,” “culture medium” and “culture media” refer to a composition for culturing cells that contains nutrients to maintain cell viability, support proliferation and optionally differentiation. A cell culture medium can contain one or more of the following: salt(s), buffer(s), amino acid(s), glucose or other sugar(s), antibiotic(s), serum or serum replacement, and other components such as growth factors, vitamins, etc.

In some aspects, the differentiation methods provided herein refer to a cell culture media (sometimes referred to as a “differentiation media” or “differentiation medium”) as a “base culture medium” supplemented with other components. As used herein, a “base culture medium” or “base culture media” refer to a composition that contains the minimal elements required for maintenance, growth and/or differentiation of cells in an in vitro environment. Examples of a base culture medium include, but are not limited to, Dulbecco's Modified Eagle Medium (DMEM), MEM, Iscove's Modified Dulbecco's Medium (IMDM), Glasgow's modified MEM (GMEM), DMEM/F12, Leibovitz L-15, RPMI-1640, CMRL, Ham F10, and HamF12. In some aspects, a base culture medium is supplemented with one or more other components such as amino acid(s), antibiotic(s), serum, growth factor(s), etc. Such components are well known in the art and described further herein.

In some aspects, a cell culture medium or base culture medium of the differentiation methods provided here is “essentially free” of a certain component or “does not comprise” a certain component. As used herein, the term “essentially free” refers to a culture medium that is at least 95% free, 96% free, 97% free, 98% free, 99% free or 100% free of a certain component or has an undetectable amount of a certain component, as measured by methods known in the art and described further herein. The terms “do not comprise” and “does not comprise” refer to a culture medium that does not contain a certain component, or has an undetectable amount of a certain component, as measured by methods known in the art and described further herein. In some aspects, a culture medium does not comprise or is essentially free of bone morphogenetic protein 4 (BMP4).

In some aspects, differentiation methods of present disclosure include culturing cells under “normoxic” or “normal oxygen” conditions. Normoxic conditions typically include culturing cells in vitro at oxygen levels of approximately 15%-20% in the air.

In other aspects, differentiation methods of the present disclosure include culturing cells under “hypoxic” or “hypoxia” conditions. Hypoxia conditions generally include culturing cells in vitro at oxygen levels of approximately 10% or less, approximately 5% or less, or approximately 1% or less, depending on the type of cell. Hypoxia conditions can be created and maintained by using a culture apparatus that allows one to control ambient gas concentrations, for example, an anaerobic chamber. Unless hypoxia conditions are specified, it can be assumed that the culturing conditions in the present disclosure are under normoxic conditions. In some aspects, cells of the present disclosure can be cultured in normoxic conditions and then in hypoxia conditions, and vice versa, using methods known in the art and described further herein.

In some aspects, the differentiation methods provided herein include certain cell culturing conditions, such passaging cells in certain culture media. As used herein, the terms “passage,” “passaged,” and “passaging” refer to the act of subdividing and plating cells at a lower concentration into one or more cell culture surfaces or vessels, when the cells have proliferated to a desired extent. Passaging typically involves detaching cells by mechanical or enzymatic methods (e.g., incubation in Accutane®) before plating, optionally at a certain cell density. Methods for passaging cells are well known and described further herein.

In some aspects, culturing and passaging in the differentiation methods provided herein are performed with one or more substrates coated onto the cell culture surface or vessel. Such substrates include, but are not limited to, vitronectin, gelatin, laminin, fibronectin, collagen (e.g., collagen I, collagen IV, or a combination thereof), elastin, osteopontin, thrombospondin, mixtures of naturally occurring cell line-produced matrices such as Matrigel™, and synthetic or man-made surfaces such as polyamine monolayers and carboxy-terminated monolayers, or a combination thereof. Methods for coating a substrate onto a cell culture surface or vessel are well known and described further herein.

In some aspects, the differentiation methods provided herein include one or more steps where cultured cells having certain biochemical characteristics are separated. As used herein, the terms “separated” and “separating” refer to a process of isolating one or more specific cell populations from a heterogeneous mixture of cells. In some aspects, the differentiation methods provided herein include separating cells having expression of vascular endothelial (VE)-cadherin (CD144), for example, to form a culture of endothelial cells (EC). As used herein, the term “expression of CD144” includes, but is not limited to, detectable expression of CD144, CD144 expression comparable to mature endothelial cells or precursors or progenitors thereof, or CD144 expression that is higher than CD144 expression in a control cell that does not express CD144 and/or is not an endothelial cell.

Cell separation methods based on a certain biochemical characteristic are well known in the art and include, but are not limited to, affinity separation, fluorescence-activated cell sorting (FACS), density gradient centrifugation, immunodensity cell isolation, microfluidic cell sorting, buoyancy-activated cell sorting, aptamer-based cell isolation, complement depletion, and more. Techniques for affinity separation include, but are not limited to, separation using antibody-coated magnetic beads (e.g., immunomagnetic cell separation), affinity chromatography, cytotoxic agents joined to a monoclonal antibody or used in conjunction with a monoclonal antibody, e.g., complement and cytotoxins, and “panning” with an antibody attached to a solid matrix, e.g. plate, or other convenient technique. In some aspects, cells of the present differentiation methods are separated by immunomagnetic cell separation.

Some aspects of the differentiation methods provided herein include culturing cells in a base culture medium containing fibroblast growth factor 2 (FGF2). FGF2, also known as basic fibroblast growth factor or FGF-β, is a growth factor and signaling protein encoded by the FGF2 gene. It possess broad mitogenic and cell survival activities, and is involved in a variety of biological processes, including embryonic development, cell growth, morphogenesis, tissue repair, tumor growth and invasion.

In some aspects, FGF is present in the base culture medium at a concentration of from about 20 μg/mL to about 100 μg/mL, or any value or range of values thereof, including, for example, from about 40 μg/mL to about 100 μg/mL, from about 50 μg/mL to about 100 μg/mL, from about 60 μg/mL to about 100 μg/mL, from about 80 μg/mL to about 100 μg/mL, from about 20 μg/mL to about 80 μg/mL, from about 40 μg/mL to about 80 μg/mL, from about 50 μg/mL to about 80 μg/mL, from about 60 μg/mL to about 80 μg/mL, from about 50 μg/mL to about 100 μg/mL, from about 50 μg/mL to about 80 μg/mL, from about 50 μg/mL to about 60 μg/mL, from about 60 μg/mL to about 100 μg/mL, from about 60 μg/mL to about 80 μg/mL, or from about 80 μg/mL to about 100 μg/mL. In some aspects, FGF is present in the base culture medium at a concentration of about 20 μg/mL, about 40 μg/mL, about 50 μg/mL, about 60 μg/mL, about 80 μg/mL or about 100 μg/mL. In some aspects, FGF is present in the base culture medium at a concentration of about 50 μg/mL.

Some aspects of the differentiation methods provided herein include culturing cells in a base culture medium containing vascular endothelial growth factor (VEGF). VEGF is a signaling protein that promotes the growth of new blood vessels. VEGF forms part of the mechanism that restores the blood supply to cells and tissues when they are deprived of oxygenated blood due to compromised blood circulation.

In some aspects, VEGF is present in the base culture medium at a concentration of from about 20 μg/mL to about 100 μg/mL, or any value or range of values thereof, including, for example, from about 40 μg/mL to about 100 μg/mL, from about 50 μg/mL to about 100 μg/mL, from about 60 μg/mL to about 100 μg/mL, from about 80 μg/mL to about 100 μg/mL, from about 20 μg/mL to about 80 μg/mL, from about 40 μg/mL to about 80 μg/mL, from about 50 μg/mL to about 80 μg/mL, from about 60 μg/mL to about 80 μg/mL, from about 50 μg/mL to about 100 μg/mL, from about 50 μg/mL to about 80 μg/mL, from about 50 μg/mL to about 60 μg/mL, from about 60 μg/mL to about 100 μg/mL, from about 60 μg/mL to about 80 μg/mL, or from about 80 μg/mL to about 100 μg/mL. In some aspects, VEGF is present in the base culture medium at a concentration of about 20 μg/mL, about 40 μg/mL, about 50 μg/mL, about 60 μg/mL, about 80 μg/mL or about 100 μg/mL. In some aspects, VEGF is present in the base culture medium at a concentration of about 50 μg/mL.

Some aspects of the differentiation methods provided herein include culturing cells in a base culture medium containing bone morphogenetic protein 4 (BMP4). In other aspects, the base culture medium does not comprise or is essentially free of BMP4. BMP4 stimulates differentiation of overlying ectodermal tissue and is known to stimulate bone formation in adult animals.

In some aspects, BMP4 is present in the base culture medium at a concentration of from about 20 μg/mL to about 100 μg/mL, or any value or range of values thereof, including, for example, from about 40 μg/mL to about 100 μg/mL, from about 50 μg/mL to about 100 μg/mL, from about 60 μg/mL to about 100 μg/mL, from about 80 μg/mL to about 100 μg/mL, from about 20 μg/mL to about 80 μg/mL, from about 40 μg/mL to about 80 μg/mL, from about 50 μg/mL to about 80 μg/mL, from about 60 μg/mL to about 80 μg/mL, from about 50 μg/mL to about 100 μg/mL, from about 50 μg/mL to about 80 μg/mL, from about 50 μg/mL to about 60 μg/mL, from about 60 μg/mL to about 100 μg/mL, from about 60 μg/mL to about 80 μg/mL, or from about 80 μg/mL to about 100 μg/mL. In some aspects, BMP4 is present in the base culture medium at a concentration of about 20 μg/mL, about 40 μg/mL, about 50 μg/mL, about 60 μg/mL, about 80 μg/mL or about 100 μg/mL. In some aspects, BMP4 is present in the base culture medium at a concentration of about 50 μg/mL.

In some aspects, a differentiation method provided herein comprises a step of culturing cells in a base culture medium comprising fibroblast growth factor 2 (FGF2) and vascular endothelial growth factor (VEGF), wherein the base culture medium does not comprise or is essentially free of BMP4. In some aspects, the method comprises a step of culturing cells in a base culture medium comprising FGF2 and VEGF for about 1 day, about 2 days, about 3 days, about 4 days or about 5 days, wherein the base culture medium does not comprise or is essentially free of BMP4. In some aspects, the culturing is on a collagen-coated (e.g., collagen IV-coated) surface.

Some aspects of the differentiation methods provided herein include culturing cells (e.g., PSC or iPSC) in a base culture medium containing a Rho associated kinase (ROCK) inhibitor. ROCK is a serine/threonine kinase that serves downstream effectors of Rho kinases, of which three isoforms exist (RhoA, RhoB and RhoC). A “ROCK inhibitor” can, for example, decrease ROCK expression and/or ROCK activity. Examples of a ROCK inhibitor include, but are not limited to, polynucleotides, polypeptides, and small molecules. More specific examples of a ROCK inhibitor include, but are not limited to, an anti-ROCK antibody, and dominant-negative ROCK variant, siRNA, shRNA, miRNA and antisense nucleic acids that target ROCK. Other examples of a ROCK inhibitor include, but are not limited to, thiazovivin, Y-27632, Fasudil, AR122-86, Y-30141, WF-536, HA-1077, hydroxyl-HA-1077, GSK269962A, SB-772077-B, N-(4-Pyridyl)-N′-(2,4,6-trichlorophenyl)urea, 3-(4-Pyridyl)-1H-indole, (R)-(+)-trans-N-(4-Pyridyl)-4-(1-aminoethyl)-cyclohexanecarboxamide, and ROCK inhibitors disclosed in U.S. Pat. No. 8,044,201, which is herein incorporated by reference in its entirety. In some aspects, the ROCK inhibitor is Y-27632.

In some aspects, the ROCK inhibitor (e.g., Y-27632) is present in the base culture medium at a concentration of from about 1 μM to about 20 μM, or any value or range of values thereof, including, for example, from about 1 μM to about 15 μM, from about 1 μM to about 10 μM, from about 1 μM to about 5 μM, from about 5 μM to about 20 μM, from about 5 μM to about 15 μM, from about 5 μM to about 10 μM, from about 10 μM to about 20 μM, from about 10 μM to about 15 μM, or from about 15 μM to about 20 μM. In some aspects, the ROCK inhibitor (e.g., Y-27632) is present in the base culture medium at a concentration of about 1 μM, about 5 μM, about 10 μM, about 15 μM, or about 20 μM. In some aspects the ROCK inhibitor (e.g., Y-27632) is present in the base culture medium at a concentration of about 10 μM.

Some aspects of the differentiation methods provided herein include culturing cells in a base culture medium containing a glycogen synthase kinase 3 (GSK3) inhibitor. GSK3 is a serine/threonine protein kinase that mediates the addition of phosphate molecules on certain serine and threonine amino acids of cellular substrates (e.g., glycogen synthase). This phosphorylation typically results in inhibition of the substrates. GSK3 has also been implicated in the control of cellular response to damaged DNA and Wnt signaling and phosphorylation of Ci in the Hedgehog (Hh) pathway, targeting it for proteolysis to an inactive form.

As used herein, a “GSK3 inhibitor” refers to a compound that inhibits one or more GSK3 enzymes. The family of GSK3 enzymes is well known and a number of variants have been described (e.g., Schaffer et al., Gene, 302:73-81, 2003). Specific examples of GSK3 inhibitors include, but are not limited to, Kenpaullone, 1-Azakenpaullone, CHIR99021, CHIR98014, AR-A014418, CT99021, CT20026, SB415286, SB216763, AR-A014418, lithium, SB 415286, and TDZD-8. Further exemplary GSK3 inhibitors include, but are not limited to, BIO (2′Z,3′£)-6-Bromomdirubm-3′-oxime (GSK3 Inhibitor IX); BIO-Acetoxime (2′Z,3′E)-6-Bromoindirubin-3′-acetoxime (GSK3 Inhibitor X); (5-Methyl-1H-pyrazol-3-yl)-(2-phenylquinazolin-4-yl)amine (GSK3 Inhibitor XIII); Pyridocarbazole-cyclopenadienylruthenium complex (GSK3 Inhibitor XV); TDZD-8,4-Benzyl-2-methyl-1,2,4-thiadiazolidine-3,5-dione (GSK3beta Inhibitor I); 2-Thio(3-iodobenzyl)-5-(1-pyridyl)-[1,3,4]-oxadiazole (GSK3beta Inhibitor II); OTDZT 2,4-Dibenzyl-5-oxothiadiazolidine-3-thione (GSK3beta Inhibitor III); alpha-4-Dibromoacetophenone (GSK3beta Inhibitor VII); AR-AO 14418 N-(4-Methoxybenzyl)-N′-(5-nitro-1,3-thiazol-2-yl)urea (GSK-3beta Inhibitor VIII); 3-(1-(3-Hydroxypropyl)-1H-pyrrolo[2,3-b]pyridin-3-yl]-4-pyrazin-2-yl-pyrrole-2,5-dione (GSK3beta Inhibitor XI); TWS1 19-pyrrolopyrimidine compound (GSK3beta Inhibitor XII); L803 H-KEAPP APPQSpP-NH2 or its Myristoylated form (GSK3beta Inhibitor XIII); 2-Chloro-1-(4,5-dibromo-thiophen-2-yl)-ethanone (GSK3beta Inhibitor VI); AR-AO 144-18; SB216763; and SB415286. In some aspects, the GSK3 inhibitor is CHIR99021.

In some aspects, the GSK3 inhibitor (e.g., CHIR99021) is present in the base culture medium at a concentration of from about 10 μM to about 60 μM, or any value or range of values thereof, including, for example, from about 10 μM to about 50 μM, from about 10 μM to about 40 μM, from about 10 μM to about 30 μM, from about 10 μM to about 20 μM, from about 20 μM to about 60 μM, from about 20 μM to about 50 μM, from about 20 μM to about 40 μM, from about 20 μM to about 30 μM, from about 30 μM to about 60 μM, from about 30 μM to about 50 μM, from about 30 μM to about 40 μM, from about 40 μM to about 60 μM, from about 40 μM to about 50 μM, or from about 50 μM to about 60 μM. In some aspects, the GSK3 inhibitor (e.g., CHIR99021) is present in the base culture medium at a concentration of about 10 μM, about 20 μM, about 30 μM, about 35 μM, about 36 μM, about 40 μM, about 50 μM, or about 60 μM. In some aspects, the GSK3 inhibitor (e.g., CHIR99021) is present in the base culture medium at a concentration of about 36 μM.

In some aspects, a differentiation method of the present disclosure comprises:
(i) culturing PSC (e.g., iPSC) in a base culture medium comprising a ROCK inhibitor;
(ii) culturing the cells of (i) in a base culture medium comprising a GSK3 inhibitor;
(iii) culturing the cells of (ii) in a base culture medium comprising FGF2, VEGF, and BMP4; and
(iv) culturing the cells of (iii) in a base culture medium comprising FGF2 and VEGF, wherein the culture medium does not comprise or is essentially free of BMP4.

In some aspects, a differentiation method of the present disclosure comprises:
(i) culturing PSC (e.g., iPSC) in a base culture medium comprising a ROCK inhibitor;
(ii) culturing the cells of (i) in a base culture medium comprising a GSK3 inhibitor;
(iii) culturing the cells of (ii) in a base culture medium comprising FGF2, VEGF, and BMP4;
(iv) culturing the cells of (iii) in a base culture medium comprising FGF2 and VEGF, wherein the culture medium does not comprise or is essentially free of BMP4; and
(v) separating the cells of (iv) having expression of CD144 to form endothelial cells.

In some aspects, the culturing of (i) and/or (ii) is for about 1 day, about 2 days, about 3 days, about 4 days, or about 5 days. In some aspects, the culturing of (i) and/or (ii) is for about 1 day. In some aspects, the cells are passaged between (iii) and (iv).

In some aspects, the method further comprises (vi) culturing the cells of (v) having expression of CD144 in a base culture medium comprising a transforming growth factor β (TGFβ) inhibitor. In some aspects, the culturing of (vi) is from about 3 days to about 9 days, from about 4 days to about 8 days, or from about 5 days to about 7 days. In some aspects, the culturing of (vi) is for about 3 days, about 4 days, about 5 days, about 6 days, about 7 days, about 8 days, or about 9 days.

TGFβ is a highly pleiotropic cytokine that plays an important role in wound healing, angiogenesis, immunoregulation and cancer. TGFβ inhibitors include, but are not limited to, any inhibitors of TGF signaling in general or inhibitors specific for TGFβ receptor (e.g., ALK5) inhibitors, which can include antibodies to, dominant-negative variants of, and siRNA and antisense nucleic acids that suppress expression of TGFβ receptors. Examples of TGFβ inhibitors include, but are not limited to, SB431542, A-83-01 (also known as 3-(6-Methyl-2-pyridinyl)-N-phenyl-4-(4-quinolinyl)-1H-pyrazole-1-carbothioamide), 2-(3-(6-Methylpyridin-2-yl)-1H-pyrazol-4-yl)-1, 5-naphthyridine, Wnt3a/BIO, BMP4, GW788388 (-4-[3-(pyridin-2-yl)-1H-pyrazol-4-yl]pyridm-2-yl}-N-(tetrahydro-2H-pyran-4-yl)benzamide), SMI 6, 3-45-(6-methylpyridin-2-yl)-4-(quinoxalin-6-yl)-1H-imidazol-2-yl)methyl)benzami, GW6604 (2-phenyl-4-(3-pyridin-2-yl-1H-pyrazol-4-yl)pyridine), SB-505124 (2-(5-benzo[1,3]dioxol-5-yl-2-tert-butyl-3H-imidazol-4-yl)-6-methylpyridine hydrochloride), SU5416, lerdelimumb (CAT-152); metelimumab (CAT-192); GC-1008; IDI 1; AP-12009; AP-1 1014; LY550410; LY580276; LY364947; LY2109761; SB-431542; SD-208; SM16; NPC-30345; KI26894; SB-203580; SD-093; ALX-270-448; EW-7195; SB-525334; FN-1233; SKI2162; Gleevec; 3,5,7,2′,4′-pentahydroxyfiavone (Morin); activin-M108A; P144; soluble TBR2-Fc; and pyrimidine derivatives and indolinones reported in Roth et al., 2010. In some aspects, the TGFβ inhibitor is SB431542.

In some aspects, the TGFβ inhibitor (e.g., SB431542) is present in the base culture medium at a concentration of from about 11.1M to about 20 μM, or any value or range of values thereof, including, for example, from about 5 μM to about 20 μM, from about 10 μM to about 20 μM, from about 11.1M to about 10 μM, and from about 11.1M to about 5 μM. In some aspects, the TGFβ inhibitor (e.g., SB431542) is present in the base culture medium at a concentration of about 1 μM, about 5 μM, about 10 μM, or about 20 μM. In some aspects, the TGFβ inhibitor (e.g., SB431542) is present in the base culture medium at a concentration of about 10 μM.

In other aspects, a differentiation method of the present disclosure comprises:
(i) culturing PSC (e.g., iPSC) on a collagen-coated surface in a base culture medium comprising a ROCK inhibitor;
(ii) culturing the cells of (i) on a collagen-coated surface in a base culture medium comprising a GSK3 inhibitor;
(iii) culturing the cells of (ii) on a collagen-coated surface in a base culture medium comprising FGF2, VEGF, and BMP4; and
(iv) culturing the cells of (iii) on a collagen-coated surface in a base culture medium comprising FGF2 and VEGF, wherein the culture medium does not comprise or is essentially free of BMP4.

In some aspects, a differentiation method of the present disclosure comprises:
(i) culturing PSC (e.g., iPSC) on a collagen-coated surface in a base culture medium comprising a ROCK inhibitor;
(ii) culturing the cells of (i) on a collagen-coated surface in a base culture medium comprising a GSK3 inhibitor;
(iii) culturing the cells of (ii) on a collagen-coated surface in a base culture medium comprising FGF2, VEGF, and BMP4;
(iv) culturing the cells of (iii) on a collagen-coated surface in a base culture medium comprising FGF2 and VEGF, wherein the culture medium does not comprise or is essentially free of BMP4; and
(v) separating the cells of (iv) having expression of CD144 to form endothelial cells.

In some aspects, the collagen is collagen IV. In some aspects, the method further comprises (vi) culturing the cells of (v) having expression of CD144 in a base culture medium comprising a transforming growth factor β (TGFβ) inhibitor. In some aspects, the culturing of (i) and/or (ii) is for about 1 day, about 2 days, about 3 days, about 4 days, or about 5 days. In some aspects, the culturing of (i) and/or (ii) is for about 1 day. In some aspects, the cells are passaged between (iii) and (iv). In some aspects, the cells are passaged between (iii) and (iv). In some aspects, the culturing of (vi) is from about 3 days to about 9 days, from about 4 days to about 8 days, or from about 5 days to about 7 days. In some aspects, the culturing of (vi) is for about 3 days, about 4 days, about 5 days, about 6 days, about 7 days, about 8 days, or about 9 days.

In some aspects, a differentiation method of the present disclosure comprises:
(i) culturing PSC (e.g., iPSC) on a collagen-coated surface in a base culture medium comprising a ROCK inhibitor;
(ii) culturing the cells of (i) on a collagen-coated surface in a base culture medium comprising a GSK3 inhibitor;
(iii) culturing the cells of (ii) on a collagen-coated surface in a base culture medium comprising FGF2, VEGF, and BMP4 for about 1 day, about 2 days, about 3 days, about 4 days, about 5 days, about 6 days, about 7 days, about 8 days, about 9 days, or about 10 days; and
(iv) culturing the cells of (iii) on a collagen-coated surface in a base culture medium comprising FGF2 and VEGF for about 1 day, about 2 days, about 3 days, about 4 days, or about 5 days, wherein the culture medium does not comprise or is essentially free of BMP4.

In some aspects, a differentiation method of the present disclosure comprises:
(i) culturing PSC (e.g., iPSC) on a collagen-coated surface in a base culture medium comprising a ROCK inhibitor;
(ii) culturing the cells of (i) on a collagen-coated surface in a base culture medium comprising a GSK3 inhibitor;
(iii) culturing the cells of (ii) on a collagen-coated surface in a base culture medium comprising FGF2, VEGF, and BMP4 for about 1 day, about 2 days, about 3 days, about 4 days, about 5 days, about 6 days, about 7 days, about 8 days, about 9 days, or about 10 days;
(iv) culturing the cells of (iii) on a collagen-coated surface in a base culture medium comprising FGF2 and VEGF for about 1 day, about 2 days, about 3 days, about 4 days, or about 5 days, wherein the culture medium does not comprise or is essentially free of BMP4; and
(v) separating the cells of (iv) having expression of CD144 to form endothelial cells.

In some aspects, the collagen is collagen IV. In some aspects, the method further comprises (vi) culturing the cells of (v) having expression of CD144 in a base culture medium comprising a TGFβ inhibitor. In some aspects, the culturing of (i) and/or (ii) is for about 1 day, about 2 days, about 3 days, about 4 days, or about 5 days. In some aspects, the culturing of (i) and/or (ii) is for about 1 day. In some aspects, the cells are passaged between (iii) and (iv). In some aspects, the cells are passaged between (iii) and (iv). In some aspects, the culturing of (vi) is from about 3 days to about 9 days, from about 4 days to about 8 days, or from about 5 days to about 7 days. In some aspects, the culturing of (vi) is for about 3 days, about 4 days, about 5 days, about 6 days, about 7 days, about 8 days, or about 9 days.

In some aspects, a differentiation method of the present disclosure comprises:
(i) culturing PSC (e.g., iPSC) on a collagen IV-coated surface in a base culture medium comprising a ROCK inhibitor;
(ii) culturing the cells of (i) on a collagen IV-coated surface in a base culture medium comprising a GSK3 inhibitor;
(iii) culturing the cells of (ii) on a collagen IV-coated surface in a base culture medium comprising FGF2, VEGF, and BMP4 for about 4 days; and
(iv) culturing the cells of (iii) on a collagen IV-coated surface in a base culture medium comprising FGF2 and VEGF for about 2 days, wherein the culture medium does not comprise or is essentially free of BMP4.

In some aspects, a differentiation method of the present disclosure comprises:
(i) culturing PSC (e.g., iPSC) on a collagen IV-coated surface in a base culture medium comprising a ROCK inhibitor;
(ii) culturing the cells of (i) on a collagen IV-coated surface in a base culture medium comprising a GSK3 inhibitor;
(iii) culturing the cells of (ii) on a collagen IV-coated surface in a base culture medium comprising FGF2, VEGF, and BMP4 for about 4 days;
(iv) culturing the cells of (iii) on a collagen IV-coated surface in a base culture medium comprising FGF2 and VEGF for about 2 days, wherein the culture medium does not comprise or is essentially free of BMP4; and
(v) separating the cells of (iv) having expression of CD144 to form endothelial cells.

In some aspects, the method further comprises (vi) culturing the cells of (v) having expression of CD144 in a base culture medium comprising a TGFβ inhibitor. In some aspects, the culturing of (i) and/or (ii) is for about 1 day, about 2 days, about 3 days, about 4 days, or about 5 days. In some aspects, the culturing of (i) and/or (ii) is for about 1 day. In some aspects, the cells are passaged between (iii) and (iv). In some aspects, the cells are passaged between (iii) and (iv). In some aspects, the culturing of (vi) is from about 3 days to about 9 days, from about 4 days to about 8 days, or from about 5 days to about 7 days. In some aspects, the culturing of (vi) is for about 3 days, about 4 days, about 5 days, about 6 days, about 7 days, about 8 days, or about 9 days.

In another aspect, a differentiation method of the present disclosure comprises a step of culturing cells having expression of CD144 in a base culture medium comprising a TGFβ inhibitor. In some aspects, the culturing is on a collagen-coated surface. In some aspects, the culturing is on a collagen I-coated surface.

In some aspects, a differentiation method of the present disclosure comprises:
(i) culturing PSC (e.g., iPSC) in a base culture medium comprising a ROCK inhibitor;
(ii) culturing the cells of (i) in a base culture medium comprising a GSK3 inhibitor;
(iii) culturing the cells of (ii) in a base culture medium comprising FGF2, VEGF, and BMP4; and
(iv) culturing the cells of (iii) in a base culture medium comprising a TGFβ inhibitor.

In some aspects, a differentiation method of the present disclosure comprises:
(i) culturing PSC (e.g., iPSC) in a base culture medium comprising a ROCK inhibitor;
(ii) culturing the cells of (i) in a base culture medium comprising a GSK3 inhibitor;
(iii) culturing the cells of (ii) in a base culture medium comprising FGF2, VEGF, and BMP4;
(iv) separating the cells of (iii) having expression of CD144; and
(v) culturing the cells of (iv) having expression of CD144 in a base culture medium comprising a TGFβ inhibitor.

In some aspects, the culturing of (i) and/or (ii) is for about 1 day, about 2 days, about 3 days, about 4 days, or about 5 days. In some aspects, the culturing of (i) and/or (ii) is for about 1 day. In some aspects, the culturing of (iii) is from about 1 day to about 10 days or from about 4 days to about 6 days. In some aspects, the culturing of (iii) is about 4 days, about 5 days, or about 6 days. In some aspects, the cells are passaged between (iii) and (iv). In some aspects, the culturing of (v) is from about 3 days to about 9 days, from about 4 days to about 8 days, or from about 5 days to about 7 days. In some aspects, the culturing of (v) is for about 3 days, about 4 days, about 5 days, about 6 days, about 7 days, about 8 days, or about 9 days.

In some aspects, a differentiation method of the present disclosure comprises:
(i) culturing PSC (e.g., iPSC) on a collagen-coated surface in a base culture medium comprising a ROCK inhibitor;
(ii) culturing the cells of (i) on a collagen-coated surface in a base culture medium comprising a GSK3 inhibitor;
(iii) culturing the cells of (ii) on a collagen-coated surface in a base culture medium comprising FGF2, VEGF, and BMP4; and
(iv) culturing the cells of (iii) on a collagen-coated surface in a base culture medium comprising a TGFβ inhibitor.

In some aspects, a differentiation method of the present disclosure comprises:
(i) culturing PSC (e.g., iPSC) on a collagen-coated surface in a base culture medium comprising a ROCK inhibitor;
(ii) culturing the cells of (i) on a collagen-coated surface in a base culture medium comprising a GSK3 inhibitor;
(iii) culturing the cells of (ii) on a collagen-coated surface in a base culture medium comprising FGF2, VEGF, and BMP4;
(iv) separating the cells of (iii) having expression of CD144; and
(v) culturing the cells of (iv) having expression of CD144 on a collagen coated surface in a base culture medium comprising a TGFβ inhibitor.

In some aspects, the collagen is collagen I or collagen IV. In some aspects, the collagen of (i) is collagen IV. In some aspects, the collagen of (ii) is collagen IV. In some aspects, the collagen of (iii) is collagen IV. In some aspects, the collagen of (v) is collagen I. In some aspects, the culturing of (i) and/or (ii) is for about 1 day, about 2 days, about 3 days, about 4 days, or about 5 days. In some aspects, the culturing of (i) and/or (ii) is for about 1 day. In some aspects, the culturing of (iii) is from about 1 day to about 10 days or from about 4 days to about 6 days. In some aspects, the culturing of (iii) is about 4 days, about 5 days, or about 6 days. In some aspects, the cells are passaged between (iii) and (iv). In some aspects, the culturing of (v) is from about 3 days to about 9 days, from about 4 days to about 8 days, or from about 5 days to about 7 days. In some aspects, the culturing of (v) is for about 3 days, about 4 days, about 5 days, about 6 days, about 7 days, about 8 days, or about 9 days.

In some aspects, a differentiation method of the present disclosure comprises:
(i) culturing PSC (e.g., iPSC) on a collagen IV-coated surface in a base culture medium comprising a ROCK inhibitor;
(ii) culturing the cells of (i) on a collagen IV-coated surface in a base culture medium comprising a GSK3 inhibitor;
(iii) culturing the cells of (ii) on a collagen IV-coated surface in a base culture medium comprising FGF2, VEGF, and BMP4; and
(iv) culturing the cells of (iii) on collagen I-coated surface in a base culture medium comprising a TGFβ inhibitor.

In some aspects, a differentiation method of the present disclosure comprises:
(i) culturing PSC (e.g., iPSC) on a collagen IV-coated surface in a base culture medium comprising a ROCK inhibitor;
(ii) culturing the cells of (i) on a collagen IV-coated surface in a base culture medium comprising a GSK3 inhibitor;
(iii) culturing the cells of (ii) on a collagen IV-coated surface in a base culture medium comprising FGF2, VEGF, and BMP4;
(iv) separating the cells of (iv) having expression of CD144; and
(v) culturing the cells of (v) having expression of CD144 on a collagen I-coated surface in a base culture medium comprising a TGFβ inhibitor.

In some aspects, the culturing of (i) and/or (ii) is for about 1 day, about 2 days, about 3 days, about 4 days, or about 5 days. In some aspects, the culturing of (i) and/or (ii) is for about 1 day. In some aspects, the culturing of (iii) is from about 1 day to about 10 days or from about 4 days to about 6 days. In some aspects, the culturing of (iii) is about 4 days, about 5 days, or about 6 days. In some aspects, the cells are passaged between (iii) and (iv). In some aspects, the culturing of (v) is from about 3 days to about 9 days, from about 4 days to about 8 days, or from about 5 days to about 7 days. In some aspects, the culturing of (v) is for about 3 days, about 4 days, about 5 days, about 6 days, about 7 days, about 8 days, or about 9 days.

### III. Other Aspects

The present disclosure is also related to endothelial cells made by any of the differentiation methods disclosed herein.

The present disclosure is also related to an organioid comprising endothelial cells made by any of the differentiation methods disclosed herein. As used herein, the term “organoid” refers to a differentiated or partially differentiated, three-dimensional (3D) cellular organism derived from pluripotent stem cells (e.g., iPSC) which is self-organized by densely accumulating cells in a controlled space. Such organisms can be crafted to replicate much of the complexity of an organ, or to express selected aspects of it, for example, only producing certain types of cells. In some aspects, the organoid is a vascular graft.

Methods for maintaining differentiated endothelial cells and organoids are well known and include culturing the cells or organoids in a cell culture medium described herein and/or cryopreservation. Methods for making an organoid typically include culturing cells in a three dimensional (3D) matrix under standard cell culturing conditions. Suitable 3D matrices include, but are not limited to, polymers (natural or synthetic), ceramics, or composites. A 3D matrix can be in the form of: a hydrogel, a porous 3D scaffold, a rapid-prototyping scaffold, a foam, a sponge, a mesh, microparticles, fiber-like networks, mixtures of naturally occurring cell line-produced matrices such as Matrigel™, and combinations thereof, for example, microparticle-loaded hydrogels.

The present disclosure is also related to a method of promoting neovascularization or vascular development comprising administration of endothelial cells or an organoid made by any of the differentiation methods disclosed herein.

The present disclosure is also related to a method of treating vasculitis or vasculopathy comprising administration of endothelial cells or an organoid made by any of the differentiation methods disclosed herein.

The present disclosure is also related to a method of treating a cardiovascular disease comprising administration of endothelial cells or an organoid made by any of the differentiation methods disclosed herein. In some aspects, the cardiovascular disease is coronary artery disease (CAD), heart arrhythmias, heart failure, heart valve disease, pericardial disease, cardiomyopathy (heart muscle disease) or congenital heart disease.

The present disclosure is also related to certain differentiation media.

In some aspects, a differentiation medium of the present disclosure comprises a base culture medium, FGF2, VEGF, and BMP4. In some aspects, the differentiation medium comprises a base culture medium, from about 20 μg/mL to about 100 μg/mL FGF2, from about 20 μg/mL to about 100 μg/mL VEGF, and from about 20 μg/mL to about 100 μg/mL BMP4. In some aspects, the differentiation medium comprises a base culture medium, about 50 μg/mL FGF, about 50 μg/mL VEGF, and about 50 μg/mL BMP4.

In some aspects, a differentiation medium of the present disclosure comprises a base culture medium, FGF2, and VEGF, wherein the medium does not comprise or is essentially free of BMP4. In some aspects, the differentiation medium comprises a base culture medium, from about 20 μg/mL to about 100 μg/mL FGF2, and from about 20 μg/mL to about 100 μg/mL VEGF. In some aspects, the differentiation medium comprises a base culture medium, about 50 μg/mL FGF, and about 50 μg/mL VEGF.

### EXAMPLES

Reference is now made to the following example, which together with the above descriptions illustrate some embodiments of the invention in a non-limiting fashion.

### Example 1

### Differentiation of Endothelial Cells

An experiment was performed to differentiate endothelial cells from human induced pluripotent stem cells (iPSC) using the following protocol.

The following reagents were used:

Iscove Modified Dulbecco Media (IMDM): Gibco, 12440-053

MEM Non-Essential Amino Acids: Gibco, 11140-050

L-glutamine: Gibco, 25030-081

Monothioglycerol: Sigma, M1753

Pen/Strep: Gibco, 15140-122

BIT 9500 Serum Substitute: Stem Cell™ Technologies, 09500

Recombinant human bone morphogenetic protein 4 (BMP4): PeproTech®, 120-05et

Human fibroblast growth factor (FGF)-Basic: PeproTech®, 100-18b

Human vascular endothelial growth factor 165 (VEGF165): PeproTech®, 100-20

Human plasma fibronectin: Life Technologies/Invitrogen, 33016015

Collagen IV, Mouse: BD Biosciences, 354233 (Aliquot and Store @−70° C.)

mTeSR™1: STEMCELL™ Technologies, 05850

Accutase™: STEMCELL™ Technologies, 07920

Rho-associated, coiled-coil containing protein kinase (ROCK) inhibitor (Y-27632): Fisher Scientific, 688000

TrypLE™ Express (lx), No Phenol Red: Life Technologies/Invitrogen, 12604-013

Human CD31 Pe Wm59: BD Biosciences, 555446

CHIR99021/glycogen synthase kinase (GSK)-3β inhibitor: Stemgent™, 04-0004-02

iPSC were obtained from Harvard Stem Cell Institute, and maintained in media made by combining about 400 mL of mTeSR™1 basal medium with about 100 mL mTeSR™1 5× supplement. Media was stored at 4 mL aliquots at −20° C. prior to use and thawed at room temperature prior to use. iPSC were passaged with Accutase™ and plated at 1:x ratio every x days onto Geltrex®-coated 10 cm² dishes.

Collagen IV (ColIV) was thawed at 4° C. and re-solubilized by vigorously vortexing for 10-15 seconds. ColIV was then diluted in ice-cold filtered 0.05 N hydrogen chloride (HCl) to make a final stock concentration of 20 μg/ml ColIV in 0.05 N HCl. Approximately 1.5 mL of diluted ColIV solution was then added in each well of 6-well plates. The plates were then incubated at 37° C. for about 2 hours and rinsed with sterile Dulbecco's Phosphate-Buffered Saline (DPBS) three times immediately before seeding.

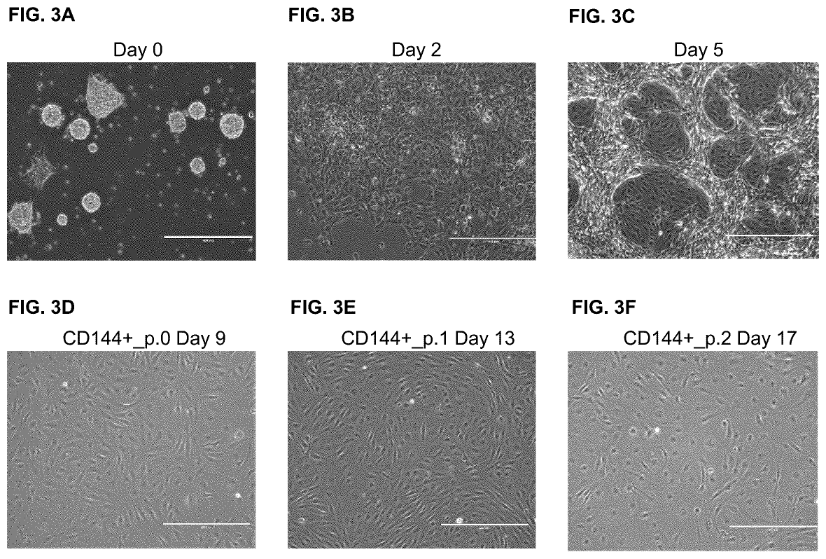

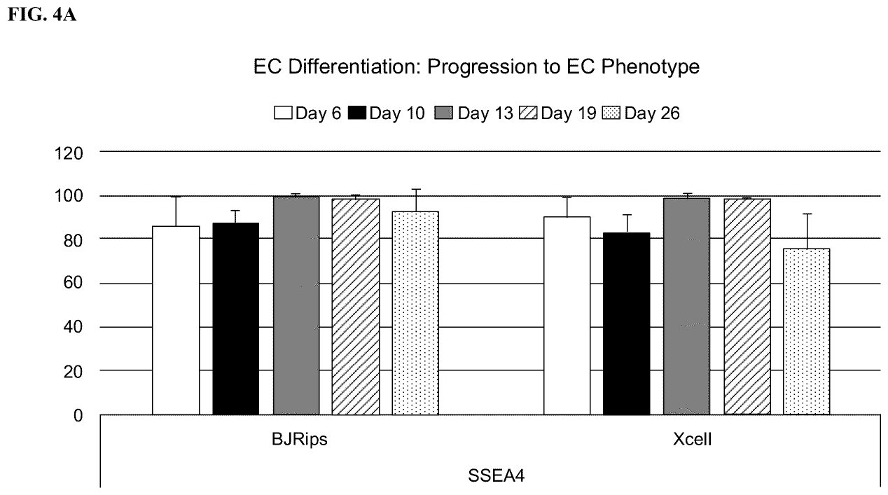

iPSC were split by adding approximately 1.5 mL Accutase™ to each well and incubating at 37° C. for 5-7 minutes. After centrifugation, the iPSC cell pellet was resuspended by adding 5 mL warm mTeSR1™ media with 10 μM Y-27632 to create a single cell suspension. iPSC were then seeded onto ColIV-coated plates at 20,000 cells/cm². The total volume of each well of a 6-well plate was approximately 2 mL. Cells were then incubated at 37° C., 4% O₂ and 5% CO₂. An exemplary image of cells at this stage is shown in FIG. 3A.

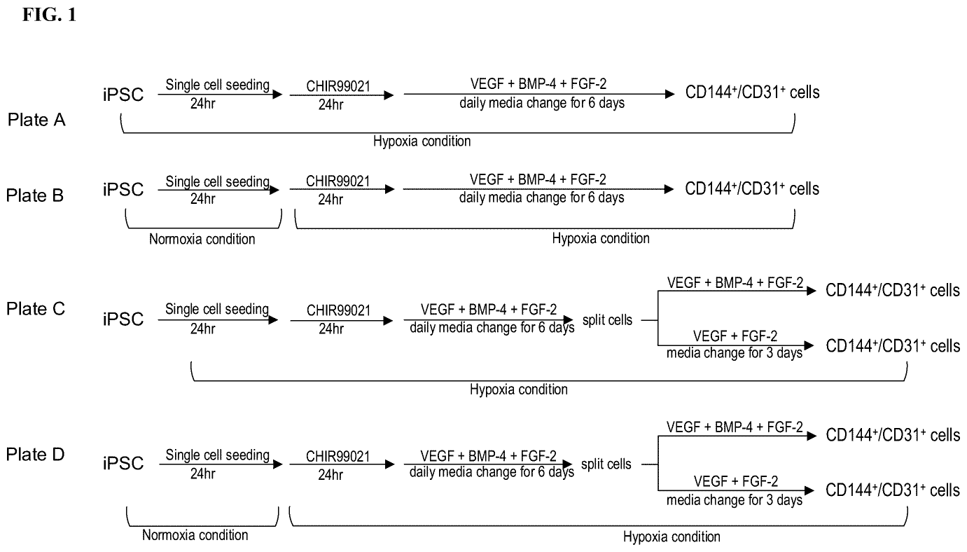

A series of differentiation experiments were then performed (FIG. 1). Plate A was grown in hypoxia conditions and differentiated over 6 days in media containing VEGF, BMP4 and FGF2. Plate B was grown in normoxia conditions for the first 24 hours, followed by 7 days of differentiation in media containing VEGF, BMP4 and FGF2. Plate C was grown in hypoxia conditions and cultured in media containing VEGF, BMP4 and FGF2 for 4 days. The cells were then split and grown in either media containing VEGF, BMP4 and FGF2 or in media containing VEGF and FGF2 for 3 days. Plate D was grown in normoxia conditions for the first 24 hours, then grown in hypoxia conditions and media containing VEGF, BMP4 and FGF2 for 4 days. The cells were then split and grown in either media containing VEGF, BMP4 and FGF2 or in media containing VEGF and FGF2 for 3 days. All conditions underwent single cell seeding and CHIR99021 exposure for 24 hours. Human pulmonary artery endothelial cells (HPAEC) were used as a positive control.

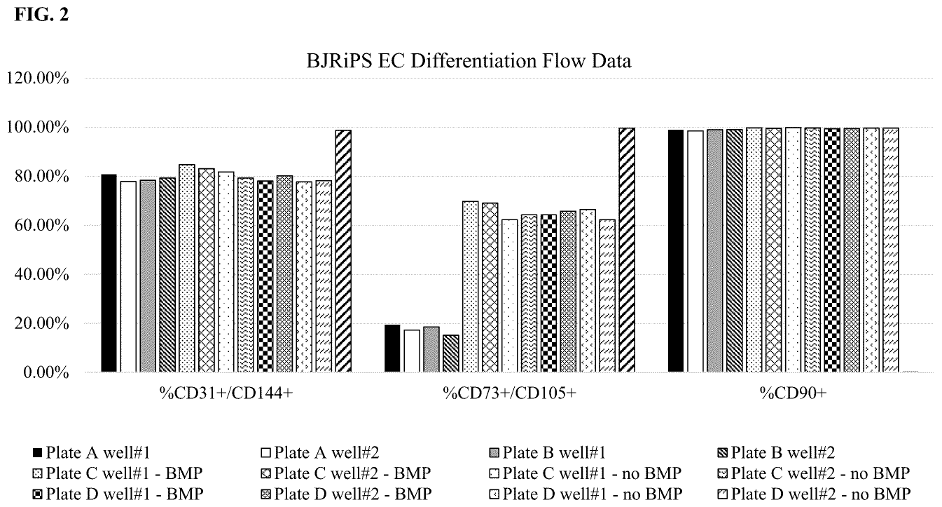

Splitting cells on day 4 did not negatively affect iPSC to endothelial cell (EC) differentiation based on canonical EC markers (CD31/CD144). However, splitting on day 4 unexpectedly produced a slightly more mature EC phenotype of CD73/CD105, and resulted in higher cell viability upon harvest and flow staining. Results of the flow staining are shown in FIG. 2, and a summary of cell viability and yield is shown in Table 1. Additionally, splitting cells on day 4 gave at least a 3-fold increase in cell number without sacrificing % CD144 for downstream magnetic isolation. Based on this data, the conditions from plate C were used for the next experiments.

TABLE 1
Cell Viability and Yield

| Sample | % Viability | Yield at Harvest |
|---|---|---|
| Plate A | 78.15% | 4.50E+06 cells |
| Plate B | 81.70% | 4.86E+06 cells |
| Plate C - BMP4 | 93.75% | 16E+06 cells |
| Plate C - no BMP4 | 93.90% | 14.7E+06 cells |
| Plate D - BMP4 | 91.65% | 13.9E+06 cells |
| Plate D - no BMP4 | 89.15% | 13.1E+06 cells |

Day 1 of iPSC to EC Differentiation

One day after iPSC were seeded on collagen IV (ColIV)-coated plates, mTeSR1™ with 36 μM CHIR99021 was added to the existing media in each well to reach a final concentration of 12 μM. Cells were then incubated at 37° C., 4% 02 and 5% CO₂. Cells were whole well-imaged using the Incucyte® Live-Cell Analysis System daily during and after differentiation.

Days 2-5 of iPSC to EC Differentiation

Cell media was replenished daily using EC media comprising Basal Differentiation Medium (BD Media), recombinant human bone morphogenetic protein 4 (BMP4), human basic fibroblast growth factor (bFGF), and human vascular endothelial growth factor 165 (VEGF165). BD media was composed of 400 mL IMDM, 100 mL BIT 9500 serum substitute, 5 mL non-essential amino acids (Thermo Fisher Scientific 11-140-050), 450 μM monothioglycerol, 2 mM GlutaMAX™ (Thermo Fisher Scientific), and 100 μg/mL Primocin® (InvivoGen). Cells were cultured in an incubator at 37° C., 4% 02 and 5% CO₂. Exemplary images of the cells at this stage are shown in FIG. 3B and FIG. 3C.

EC are characterized by expression of the surface protein markers platelet endothelial cell adhesion molecule (PECAM/CD31) and vascular endothelial cadherin (VEcadherin/CD144). EC also express von Willebrand Factor (vWF), vascular endothelial growth factor receptor 2 (Flk-/VEGFR-2/KDR), vascular endothelial growth factor receptor 1 (Flt-1/VEGFR1), and endothelial nitric oxide synthase (eNOS). The in vitro functional abilities of EC include uptake of low density lipoproteins (LDLs) and lectin binding.

Day 6 of iPSC to EC Differentiation—Preparation of Cells for CD144+ Magnetic Selection

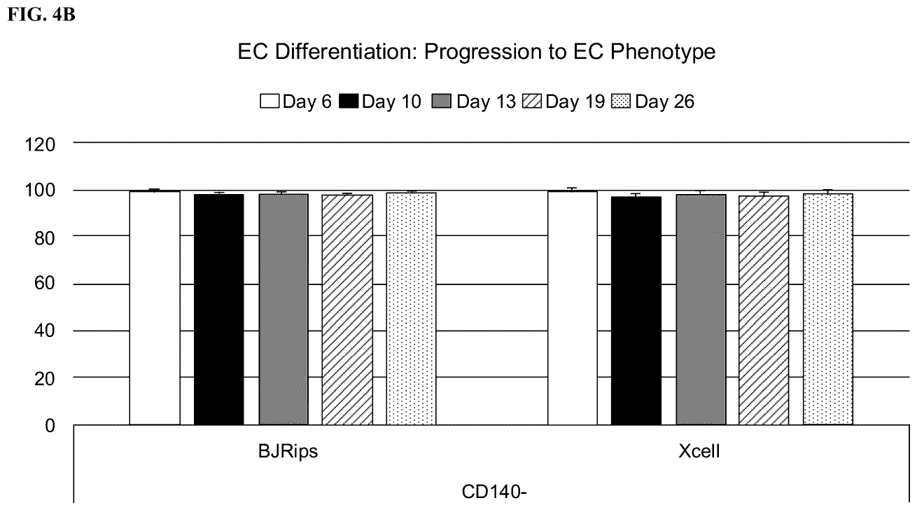

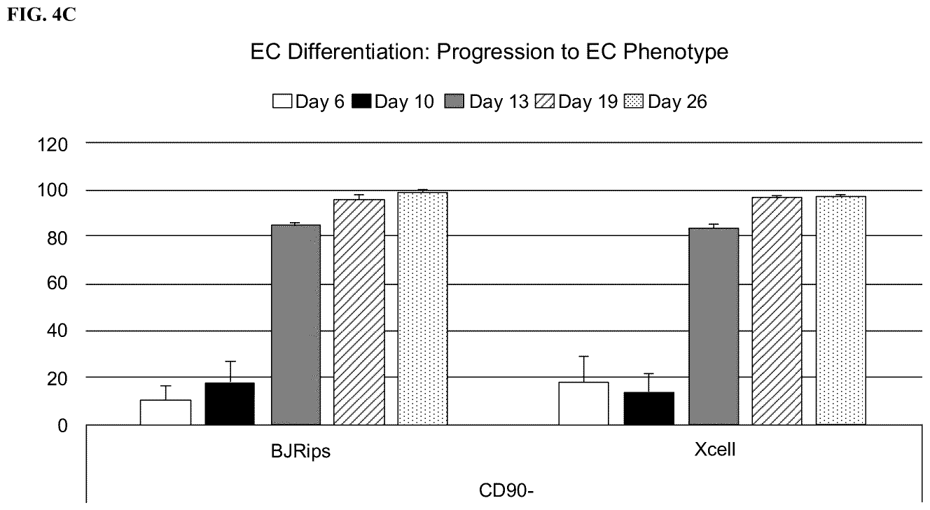

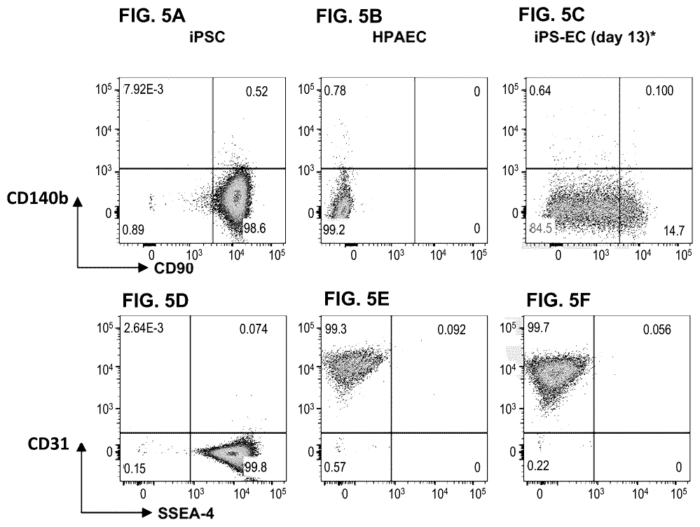

Selection of CD144+ cells was used to determine the success of EC differentiation from iPSC using this protocol. Cells were harvested and rinsed with DPBS 3 times. An appropriate volume of TrypLE™ was added, and cells were incubated for 5-7 minutes at room temperature. Detached cells were collected and pelleted at 200×g for 5 minutes. Cells were then counted and used for flow cytometry analysis. Approximately 4E6 cells were transferred to a 15 mL centrifuge for flow cytometry analysis of endothelial protein expression (CD144, CD31), mesenchymal progenitors (CD140b), and stem cell marker expression (SSEA4). Results from this analysis are shown in FIG. 4A-4C and FIG. 5A-5F.

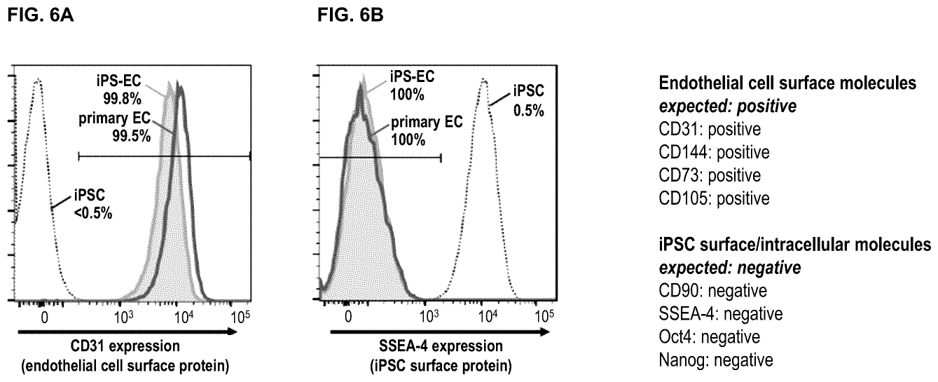

250,000 cells per flow cytometry tube/sample were used for the analysis. The remaining cells were centrifuged at 200×g for 5 minutes. The cells were then resuspended in running buffer at 10E7 cells/80 μL in sterile filtered running buffer comprising PBS (Gibco), 25 mM HEPES (4-(2-hydroxyethyl)-1-piperazineethanesulfonic acid; Gibco) and 0.5% bovine serum albumin (BSA)(Miltenyi cat #130-091-376)). CD144 microbeads (Miltenyi cat #130-097-857) were then added and mixed with running buffer suspension at 20 μL per 10E7 cell followed by 15 minutes of incubation at 4° C. Cells were then washed with 2 mL of running buffer per 10E7 cells and pelleted at 300×g for 5 minutes. Pellets were then resuspended in running buffer at 25E6 cells/mL. Cells were then ran on the Miltenyi Biotec AutoMACS® Pro system for selection of CD144+ cells according to Miltenyi AutoMACS® Pro System CD144 microbead protocol. Results from this analysis are shown in FIG. 6A-6B.

Day 7-9/10 of iPSC-EC Differentiation: E4 Step to PO Step

Media was carefully removed and warm EC expansion media was added [EGM2 media (Lonza CC-3162), supplemented with 10 μM SB431542 (Reprocell 4001010), 100 μg/mL Primocin® (InvivoGen ant-pm-2), and 16.2% FBS (VWR 76294-180)]. Cells were cultured in a normoxia incubator at 37° C., 20% 02, and 5% CO₂. Media was removed and replaced approximately every 48 hours.

Day 9/10 of iPSC-EC Differentiation: Harvest of iPSC-EC PO Cells

At approximately 24 hours from previous media change, cells were harvested by first rinsing cells with DPBS 3 times. Accutase™ was added and the cells were incubated for 5 minutes at 37° C. Once detached, cells were transferred to a centrifuge tube. Cell culture vessels were rinsed twice using BD media, which was added to the centrifuge tube.

The cell-containing media was then filtered using 30 μM filters and cells were pelleted by centrifugation at 200×g for 5 minutes, with maximum acceleration and deceleration at 8° C. Cell pellets were resuspended in 5 mL of EC expansion media. Total cell count, cells/ml and percentage viability was then determined for use in quality control testing.

Cells were imaged every 12 hours from day 0 and demonstrated to have cobblestone morphology at terminal differentiation. Exemplary images of cells from day 9, day 13 and day 17 are shown in FIG. 3D-3F.

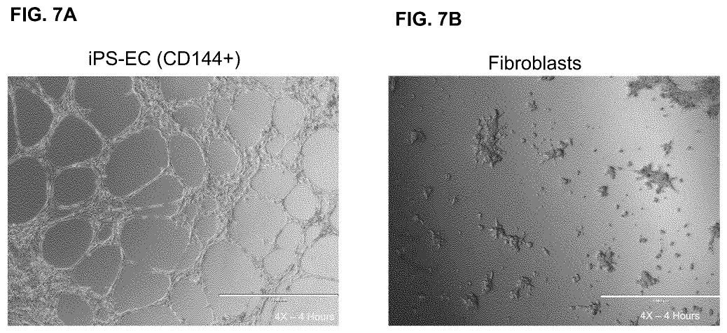

Differentiated cells were further characterized with a tube formation assay using the following protocol. Test differentiated cells were co-cultured with or without 1 μM imatinib mesylate to inhibit angiogenesis, and tube formation was observed. Specifically, the wells of 96-well plates (Costar, Product No. 387) were coated with 30 μL of Matrigel® Growth Factor Reduced (GFR) Basement Membrane Matrix (Corning®). Plates were spun for 1 minute at 1,500 rotations per minute (RPM) and incubated for 30 minutes at 30° C. Cells were then lifted from culture plates, counted and resuspended in Dulbecco's Modified Eagle Medium (DMEM)/F12 media such that 30 μL of media was available for each well. Each well received 30,000 test differentiated cells. For the tube formation assay, after gelling, 30 μL of cell suspension was carefully placed on top. Plates were loaded into the Incucyte® Live-Cell Analysis System and images were taken every hour. Exemplary images of the resulting differentiated cells are shown in FIG. 7A-7B. Differentiated cells were found to rearrange in a tubular structure resembling angiogenesis sprouting, and angiogenesis capability was confirmed.

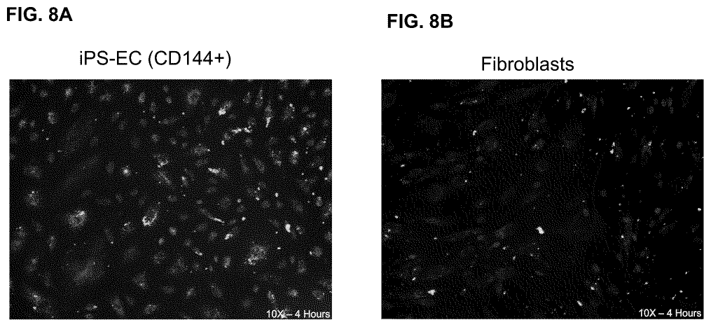

Differentiated cells were further characterized using an acetylated low density lipoprotein (Ac-LDL) assay. This assay is based on the principle that “scavenger” receptors on endothelial cells can bind and uptake Ac-LDL. Test differentiated cells were harvested, counted and seeded at 5,000-10,000 cells per well in 96-well plates coated with Collagen I. Human umbilical vein endothelial cells (HUVEC) and fibroblasts were used as positive and negative controls, respectively. Cells were then incubated overnight at 37° C. The media was replaced with 100 μL (+/−) 10 μg/mL Ac-LDL and 1:2000 dilution of nuclear stain, NucLight Rapid Red (non-perturbing, cell permeable). Cells were incubated at 37° C. followed by PBS washes. Cells were then imaged on Incucyte® S3 every 30 minutes at 20× magnification. Exemplary images of the resulting differentiated cells are shown in FIG. 8A-8B. The data suggests that iPSC derived ECs (CD144+BJRIP38) internalize Ac-LDL via receptor mediated endocytosis and have marked Ac-LDL update.

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for differentiating pluripotent stem cells into endothelial cells, comprising:
(i) culturing pluripotent stem cells on a collagen IV-coated surface in a base culture medium comprising a Rho-associated, coiled-coil containing protein kinase (ROCK) inhibitor;
(ii) culturing the cells of (i) on a collagen IV-coated surface in a base culture medium comprising a glycogen synthase kinase 3 (GSK3) inhibitor;
(iii) culturing the cells of (ii) on a collagen IV-coated surface in a base culture medium comprising fibroblast growth factor 2 (FGF2), vascular endothelial growth factor (VEGF), and bone morphogenetic protein 4 (BMP4) for about 4 days;
(iv) culturing the cells of (iii) on a collagen IV-coated surface in a base culture medium comprising FGF2 and VEGF for about 2 days, wherein the culture medium does not comprise or is essentially free of BMP4; and
(v) separating the cells of (iv) having expression of CD144 to form endothelial cells.

2. The method of claim 1, further comprising:
(vi) culturing the cells of (v) having expression of CD144 in a base culture medium comprising a transforming growth factor β (TGFβ) inhibitor.

3. A method for differentiating pluripotent stem cells into endothelial cells, comprising:
(i) culturing pluripotent stem cells on a collagen IV-coated surface in a base culture medium comprising a ROCK inhibitor;
(ii) culturing the cells of (i) on a collagen IV-coated surface in a base culture medium comprising a GSK3 inhibitor;
(iii) culturing the cells of (ii) on a collagen IV-coated surface in a base culture medium comprising FGF2, VEGF, and BMP4;
(iv) separating the cells of (iii) having expression of CD144; and
(v) culturing the cells of (iv) having expression of CD144 on a collagen I-coated surface in a base culture medium comprising a TGFβ inhibitor to form endothelial cells.

4. The method of claim 1, wherein the ROCK inhibitor is Y-27632.

5. The method of claim 4, wherein Y-27632 is present in the culture medium at a concentration of about 10 μM.

6. The method of claim 1, wherein the culturing of (i) is for about 1 day.

7. The method of claim 1, wherein the GSK3 inhibitor is CHIR99021.

8. The method of claim 7, wherein CHIR99021 is present in the culture medium at a concentration of about 36 μM.

9. The method of claim 1, wherein the culturing of (ii) is for about 1 day.

10. The method of claim 1, wherein FGF2 is present in the culture medium at a concentration of about 50 μg/mL.

11. The method of claim 1, wherein VEGF is present in the culture medium at a concentration of about 50 μg/mL.

12. The method of claim 1, wherein BMP4 is present in the culture medium at a concentration of about 50 μg/mL.

13. The method of claim 1, wherein the cells are passaged between (iii) and (iv).

14. The method of claim 3, wherein the culturing of (iii) is from about 4 days to about 6 days.

15. The method of claim 3, wherein the TGFβ inhibitor is SB431542.

16. The method of claim 15, wherein SB431542 is present in the culture medium at a concentration of about 10 μM.

17. The method of claim 2, wherein the culturing of (vi) is for about 6 days.

18. The method of claim 3, wherein the culturing of (v) is for about 6 days.

19. The method of claim 1, wherein the separating is by immunomagnetic cell separation.

20. The method of claim 1, wherein the culturing of (i) and/or (ii) are performed under hypoxia conditions.

21. The method of claim 1, wherein the pluripotent stem cells are embryonic stem cells (ESC), induced pluripotent stem cells (iPSC), embryonic germ cells, or adult stem cells.

22. Endothelial cells made by the method of claim 1.

23. An organoid comprising the endothelial cells of claim 22.
